(19) United States
(12) Patent Application Publication
(10) Pub. No.: US 2010/0137848 A1
(43) Pub. Date: Jun. 3, 2010

(54) LASER SYSTEM FOR HUMAN SKIN SURFACE TREATMENTS

(75) Inventor: Dante Marchi, Modena (IT)

Correspondence Address: PYLE & PIONTEK, LLC, 221 N LASALLE STREET , ROOM 1207, CHICAGO, IL, 60601, US

(73) Assignee: LASERING S.R.L., Modena (IT)

(21) Appl. No.: 12/451,893

(22) PCT Filed: Jun. 10, 2008
(86) PCT No.: PCT/EP2008/004622

§ 371 Date: Dec. 4, 2009

(30) Foreign Application Priority Data
Jun. 13, 2007 (IT) MO2007A000197

(51) Int. Cl. A61B 18/20

(52) U.S. Cl. 606/3; 606/9

(57) ABSTRACT

The present invention relates to a field of systems for human skin medical treatments. The present system consists of a continuous wave-operated laser source (2) and of a biomedical laser system (1) connected to a scanning device (5) by means of an articulated transmission system (4) said laser being adapted to radiate a continuous CO₂ laser beam.

It is an object of the present invention a laser system for human skin surface treatments.

The above mentioned biomedical laser system enables to perform medical treatments such as resurfacing, skin rejuvenation, spot treatments and other human skin treatments, by using a continuous wave CO₂ laser beam which passes through an intermittence apparatus and through an articulated transmission system it is conveyed to a scanning device, synchronized with the intermittence apparatus adapted to direct said intermittent CO₂ laser beam towards the patient's skin.

The techniques adopted so far for medical treatments such as skin resurfacing, skin rejuvenation, spot treatments and other human skin surface treatments by using a CO₂ laser beam and a scanning device make use of pulsed-emission sources or of continuous wave emission sources but not made intermittent by an intermittence apparatus synchronized with a scanning device; said techniques showing the following drawbacks:
poor reliability in maintaining the set power and reproducibility values for each laser pulse radiated by the pulsed-emission source;
inability of locating two spots, emitted one after the other, in non mutually consecutive positions with laser beams radiated from the continuous wave emission source.

It is to be noted that the term “spot” means the mark the CO₂ laser beam leaves on the patient's skin area hit by said beam.

It is an object of the present invention to overcome the above mentioned drawbacks.

Said aims are thoroughly achieved by the laser system for human skin surface treatments which is characterized by what stated in the appended claims.

Features and advantages will be better understood from the following description of a preferred—but not exclusive—embodiment to be considered by way of an example and not restrictive, as shown in the accompanying drawings, in which:

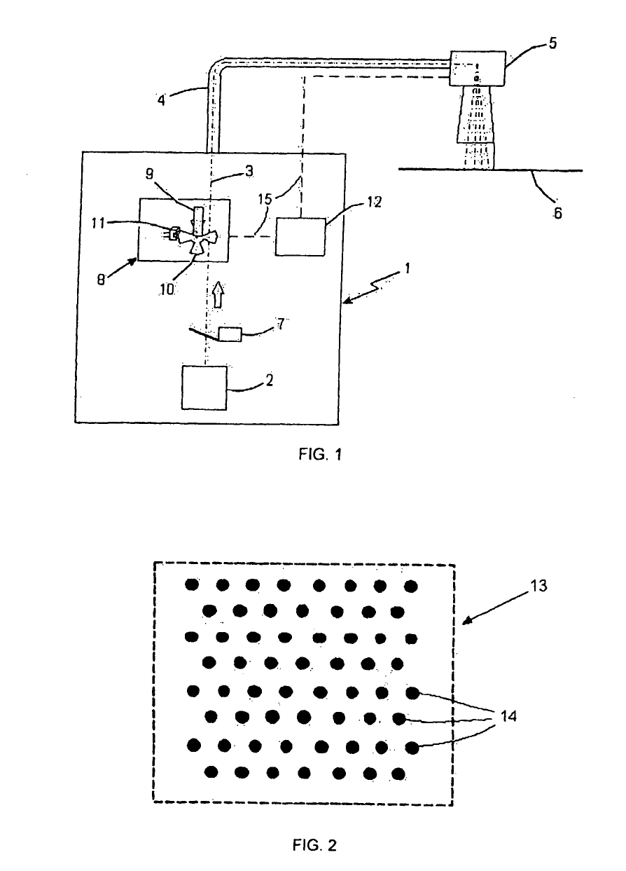

FIG. 1 is a schematic view of the functional components of the system, object of the present invention;

FIG. 2 shows the final pattern of the positioning of spots 14 within the skin area 13 to be treated.

With reference to FIG. 1, number 1 is the biomedical laser system connected, through an articulated transmission system 4 made up of either an articulated arm or by an optical fibre system, to a hand-operated scanning device 5 whose function is to direct the CO₂ laser beam 3 towards the patient's skin 6.

The laser source 2 is of a known type, operating by means of a continuous wave adapted to radiate a continuous CO₂ laser beam having a wavelength ranging from 9 μm to 12 μm; said laser source 2 and the intermittence apparatus 8 are located inside the biomedical laser system 1.

The object of the present invention proves to be advantageously operable even when the intermittence apparatus 8 is located outside the biomedical laser system 1; in this case the laser source 2 is connected to the above mentioned intermittence apparatus 8 by means of a plurality of stiff pipes adapted to allow the continuous CO₂ laser beam to go through.

The biomedical laser system 1 comprises: a general safety shutter 7 which—when activated—allows the continuous CO₂ laser beam 3 radiated from the laser source 2 to go through; the intermittence apparatus 8, which is responsible for cutting off said continuous CO₂ laser beam, made up of an electric motor 9 which rotates a slotted disk 10 with alternate solid and empty sectors.

The above mentioned intermittence apparatus further provides an optical detector 11 to detect the position of the solid and empty sectors of the slotted disk 10.

A control and guide device 12 for controlling and guiding the mobile scanning device 5, connected to such scanning device 5 and to the optical detector 11 by means of electric wiring 15, is further provided.

Now the operation of the present invention will be described.

The medical treatment starts when the operator, after activating the laser source 2, opens the general safety shutter 7; enabling the biomedical laser system 1 to supply the intermittent CO₂ laser beam 3 towards the patient's skin 6 by means of the scanning device 5.

Once the opening of the general safety shutter 7 has been activated, the continuous CO₂ laser beam 3, radiated by the laser source 2, passes through the intermittence apparatus 8 hitting the slotted disk 10 rotating as shown in FIG. 1 and accordingly turning into an intermittent CO₂ laser beam 3 whose life is proportional to the rotational speed of the slotted disk and to the number of slots of said disk.

Then such intermittent laser beam will pass through an articulated transmission system 4 consisting, for example, of an optical fibre or of a multi-mirror articulated arm, which will direct it towards the scanning device 5 adapted to radiate the above mentioned intermittent laser beam towards the patient's skin 6.

The optical detector 11, inside the intermittence apparatus 8, acts to detect the position of the solid and empty sectors of the slotted disk 10 and according to it, controls the passing or the cutting off of the laser beam accessing the blocking system (see FIG. 1). By means of an electric signal, the optical sensor 11 provides the detected position of the slotted disk 10 for the control and guide device 12 which, in turn, will synchronize the signal received to the movement of the motors, inside the scanning device, for directing the intermittent laser beam on the skin area 13 to be treated. The control and guide device will ensure the moving of the motors to reach exactly the position along the coordinates where the next spot will hit the area to be treated exclusively in the time when the laser beam is blocked by a solid sector of the above mentioned slotted disk.

As the slotted disk 10 is continuously rotated by the electric motor 9, the passage of each solid sector of said disk is obviously followed by an empty one which will cause the CO₂ laser beam 3 to go through the intermittence apparatus and to radiate towards the patient's skin 6 and again the passage of another solid sector, during which the above stated motors will position along the new coordinates of the next spot.

Clearly the skin exposure time to each CO₂ laser beam radiated from the scanning device 5 is a function of the rotational speed of the slotted disk 10 and of the number of slots in said disk.

The above mentioned sequences of motor positioning and intermittent CO₂ laser beam emissions follow one another and continue till the end of each scan, that is till the positioning of the last spot inside each skin area to be treated.

During the scanning step, the control and guide device 12 determines the coordinates of spots 14 according to a specific algorithm which takes into account the scan size, the spot diameter and the thermal relaxing of every single skin area under treatment.

Further the laser system for human skin surface treatments, object of the present invention, proves to be effective when downstream the laser source 2, in place of the intermittence apparatus 8, any element capable, at regular intervals, of blocking the continuous CO₂ laser beam 3 radiated from the laser source 2 is used; said element making use of mechanical and/or electric systems to block the continuous CO₂ laser beam.

1. A laser system for human skin surface treatments, characterized in that it comprises a continuous wave-operated laser source (2) adapted to radiate a continuous CO₂ laser beam (3); a biomedical laser system (1) connected to a scanning device (5) through an articulated transmission system (4); an intermittence apparatus (8) adapted to shield and block the continuous CO₂ laser beam (3) radiated from the laser source (2) at regular intervals; an optical sensor (11) used to detect the passing or blocking of the CO₂ laser beam and to supply the information acquired, by means of an electric signal, to the control and guide device (12) which automatically manages the positioning sequence of the spots (14) on the skin area to be treated (13) so as to move the motors of the aforementioned scanning device in order to position each of the above spots only when the slotted disk interrupts the CO₂ laser beam (3).

2. The system according to claim 1, characterized in that the intermittence system (8) consists of a slotted disk (10) provided with a plurality of slots alike and equally spaced apart and rotated by an electric motor (9).

3. The system according to claim 1, characterized in that the scanning device (5) provides the radiation of an intermittent CO₂ laser beam (3) onto the skin area to be treated (13) following a specific algorithm which takes into account the size of the above mentioned area, the spot diameter and the thermal relaxing of every single skin area under treatment.

4. The system according to claim 1, characterized in that the intermittence apparatus (8) is located inside the biomedical laser system (1).

5. The system according to claim 1, characterized in that the intermittence apparatus (8) is located outside the biomedical laser system (1) and in that the laser source (2) is connected to said intermittence apparatus by means of a plurality of stiff pipes adapted to allow the passing of the continuous CO₂ laser beam (3).

6. The system according to claim 1, characterized in that downstream the laser source (2) in place of the intermittence apparatus (8) an element adapted, at regular intervals, to block the continuous CO₂ laser beam (3) radiated by the laser source (2) is used; said element making use of mechanical and/or electrical systems to block the continuous CO₂ laser beam (3).

7. The system according to claims claim 1, characterized in that the laser source (2) is adapted to radiate the continuous-wave CO₂ laser beam (3) having a wavelength ranging from 9 μm to 12 μm.

8. The system according to claim 3, characterized in that the laser source (2) is adapted to radiate the continuous-wave CO₂ laser beam (3) having a wavelength ranging from 9 μm to 12 μm.

9. The system according to claim 4, characterized in that the laser source (2) is adapted to radiate the continuous-wave CO₂ laser beam (3) having a wavelength ranging from 9 μm to 12 μm.

10. The system according to claim, 5 characterized in that the laser source (2) is adapted to radiate the continuous-wave CO₂ laser beam (3) having a wavelength ranging from 9 μm to 12 μm.

11. The system according to claim 6 characterized in that the laser source (2) is adapted to radiate the continuous-wave CO₂ laser beam (3) having a wavelength ranging from 9 μm to 12 μm.
